(19) United States
(12) Patent Application Publication
(10) Pub. No.: US 2021/0285053 A1
(43) Pub. Date: Sep. 16, 2021

(54) L1TD1 AS PREDICTIVE BIOMARKER OF COLON CANCER

(71) Applicant: Turun yliopisto, Turun yliopisto (FI)

(72) Inventors: Deepankar CHAKROBORTY, Turku (FI); Emani Maheswara REDDY, Turku (FI); Laura ELO, Turku (FI); Riitta LAHESMAA, Turku (FI); Ari RISTIMÄKI, Helsinki (FI); Caj HAGLUND, Helsinki (FI)

(73) Assignee: Turun yliopisto, Turun yliopisto (FI)

(21) Appl. No.: 17/058,926

(22) PCT Filed: May 29, 2019
(86) PCT No.: PCT/FI2019/050416

§ 371 Date: Nov. 25, 2020

(30) Foreign Application Priority Data
May 29, 2018 (FI) 20185489

(51) Int. Cl. C12Q 1/6886

(52) U.S. Cl. CPC C12Q 1/6886; C12Q 2600/158; C12Q 2600/118

(57) ABSTRACT

The present invention relates to biomarkers, such as L1NE-1 type transposase domain containing 1 (L1TD1) as predictive prognostic markers of colon cancer. The invention also relates to a method of prognosing colon cancer, and to a kit for use in said method.

### FIELD OF THE INVENTION

The present invention relates to the field of molecular diagnostics. More specifically, the invention relates to means and methods for prognosticating colon cancer.

### BACKGROUND OF THE INVENTION

Stem cell-like gene signatures have been detected in various cancers, and embryonic stem cell factors OCT4 and NANOG have been associated with enhanced tumorigenesis and poor prognosis in various cancer types.

LINE-1 type transposase domain containing 1 (L1TD1) is an RNA-binding protein required for self-renewal of undifferentiated embryonic stem cells. Recently, L1TD1 protein was shown to form a core interaction network with OCT4, NANOG, LIN28, and SOX2 in human embryonic stem cells (hESCs), and L1TD1 depletion resulted in downregulation of OCT4, NANOG, and LIN28 in hESCs. Earlier reports have demonstrated the association of OCT4 and NANOG with poor prognosis in different cancer types.

In addition to embryonic stem cells, expression of L1TD1 has earlier been reported in the brain and colon, as well as in different cancers such as seminoma, embryonic carcinomas, medulloblastoma, and colon adenocarcinoma. L1TD1 has been shown to be essential for self-renewal of embryonal carcinoma cells and support the growth of seminoma cells. Interestingly, immunohistochemistry data from the Human Protein Atlas suggest that L1TD1 is expressed at high levels in a subset of colon cancer samples. Moreover, WO 2013/033626 and US 2010/0292094 disclose that a higher level of L1TD1 relative to control levels is indicative of colon cancer, a neoplastic large intestine cell or a cell predisposed to the onset of a neoplastic state.

Colon cancer is the third most commonly diagnosed cancer worldwide with 1.4 million new cases in 2012. Even though colorectal cancer is one of the most well-studied cancer types, there is a lack of predictive prognostic markers.

### BRIEF DESCRIPTION OF THE INVENTION

An object of the present invention is to provide improved methods and means for prognosing colon cancer in a subject.

This object is achieved by a method, use and a kit, which are characterized by what is stated in the independent claims. Some specific embodiments of the invention are disclosed in the dependent claims.

The present invention thus provides a method of prognosing colon cancer in a subject, wherein the method comprises assaying a sample obtained from said subject for the level of L1TD1 and ASRGL1 expression, and comparing the assayed levels of L1TD1 and ASRGL1 to corresponding control levels, and prognosing said colon cancer on the basis of said comparison. Also provided is use of L1TD1 and ASRGL1 in prognosing colon cancer.

In a further aspect, the invention provides a kit for use in the present method, the kit comprises one or more testing agents capable of specifically detecting the expression level of L1TD1 and ASRGL1 in a biological sample obtained from a subject whose colon cancer is to be determined.

Further aspects, specific embodiments, objects, details, and advantages of the invention are set forth in the following drawings, detailed description, and examples.

### BRIEF DESCRIPTION OF THE DRAWINGS

In the following the invention will be described in greater detail by means of preferred embodiments with reference to the attached drawings, in which

FIGS. 1A to 1C—Kaplan-Meier curves showing disease-free survival for the three colon cancer data sets. The curves present survival data for the two groups of colon cancer patients based on L1TD1 expression level (high or low). Curve with solid line curve corresponds to patients with high L1TD1 expression and curve with dotted line represents the patients with low L1TD1 expression. The x-axis shows disease-free survival time in years and the y-axis shows the probability of disease-free survival. The risk table shows the number of patients at risk at the given time point.

FIG. 2—Heatmaps showing signed P-value of Spearman rank correlation for the 20 most significantly co-expressed interaction partners of L1TD1 determined on the basis of the seminoma and stem cell data sets; co-expression in (A) seminoma and stem cell data sets, and (B) colon cancer data sets. The top interaction partners were selected by first ranking the interaction partners in the hESC and seminoma data sets based on descending order of Spearman rank correlation values computed for pairwise correlations between L1TD1 and the said interaction partner. Then the maximum rank over these data sets was selected as a representative statistic for each interaction partner. The list was ordered (ascending) based on this maximum rank and 20 interaction partners were selected from the top of the list. The signed P-value of Spearman rank correlation was defined as 1—P-value of Spearman rank correlation multiplied by the sign of the correlation.

FIG. 3A demonstrates that immunostaining of healthy colon cells for L1TD1 reveals organized and regulated expression of L1TD1.

FIG. 3B demonstrates immunostaining of a sample of colorectal adenocarcinoma revealing high levels of L1TD1 expression.

FIGS. 4A to 4C are Kaplan-Meier curves showing disease-free survival for the three colon cancer data sets. The curves present survival data for the three groups of colon cancer patients based on their L1TD1 and ASRGL1 expression levels: patients with no expression of L1TD1 or ASRGL1 (solid line), patients expressing only L1TD1 but not ASRGL1 (dashed line), and patients expressing L1TD1 and ASRGL1 (dotted line). The x-axis shows disease-free survival time in years and the y-axis shows the probability of disease-free survival.

FIG. 5A to 5C are Kaplan-Meier curves showing disease-free survival for the three colon cancer data sets. The curves present survival data for the three groups of colon cancer patients based on their L1TD1, ASRGL1 and RETNLB expression levels: patients with no expression of L1TD1, ASRGL1 or RETNLB (solid line), patients expressing only L1TD1 but not ASRGL1 or RETNLB (dashed line), and patients expressing L1TD1, ASRGL1 and RETNLB (dotted line). The x-axis shows disease-free survival time in years and the y-axis shows the probability of disease-free survival.

FIGS. 6A to 6C are Kaplan-Meier curves showing disease-free survival for the three colon cancer data sets. The curves present survival data for the three groups of colon cancer patients based on their L1TD1, ASRGL1, RETNLB and SPINK4 expression levels: patients with no expression of L1TD1, ASRGL1, RETNLB or SPINK4 (solid line), patients expressing only L1TD1 but not ASRGL1, RETNLB or SPINK4 (dashed line), and patients expressing L1TD1, ASRGL1, RETNLB and SPINK4 (dotted line). The x-axis shows disease-free survival time in years and the y-axis shows the probability of disease-free survival.

### DETAILED DESCRIPTION OF THE INVENTION

The invention relates to different aspects of L1TD1 as a prognostic predictive marker for colon cancer. Accordingly, in some aspects, the invention relates to different uses of said marker, and to different in vitro methods of prognosing colon cancer.

The present invention is, at least partly, based on a surprising finding that increased expression of L1TD1 in a sample obtained from a subject suffering from colon cancer indicates good prognosis.

During the course of the present invention, three independent gene-expression microarray data sets (N=1052) were analyzed. The investigators set out to examine the prognostic significance of L1TD1 in colon cancer with the hypothesis that high expression of L1TD1 would be associated with poor prognosis. Earlier reports had demonstrated the association of OCT4 and NANOG with poor prognosis in different cancer types, including medulloblastoma and seminoma. Therefore, it came as a surprise that high expression of L1TD1 was associated with positive prognosis in multiple independent colon cancer data sets.

The present findings are in contrast to an earlier study on medulloblastoma where high expression of L1TD1 was shown to be linked with poor prognosis (Santos et al., 2015, Stem Cells Dev., 24(22):2700-8). Without being limited to any theory, this difference might be explained by the lack of co-expression of L1TD1 with one or more of its top 20 interaction partners, i.e. OCT4, TRIM71, DPPA4, DNMT3B, LRPPRC, MRPS17, PARP1, RPF2, HSP90AA1, IGF2BP1, DNAJA2, NANOG, ALPL, EIF3B, NCL, LIN28A, NOLC1, CCT8, RRS1, and SFPQ (Table 1), which were identified in an earlier study by Mass spectrometry and co-immunoprecipitation (Emani et al., 2015, Stem Cell Reports 4, 519-528).

TABLE 1
Top 20 interaction partners of L1TD1

| GENE NAME | UNIOPROT ID (HUMAN) | UNIPROT ENTRY NAME | UNIPROT PROTEIN NAME |
|---|---|---|---|
| OCT4 | Q01860 | POSF1_HUMAN | POU domain, class S, transcription factor 1 |
| TRIM71 | QZQ1W2 | LIN41_HUMAN | E3 ubiquitin-protein ligase TRIM71 |
| DPPA4 | Q7L190 | DPPA4_HUMAN | Developmental pluripotency-associated protein 4 |
| DNMT3B | Q9UBC3 | DNM3B_HUMAN | DNA (cytosine-5)-methyltransferase 3B |
| LRPPRC | P42704 | LPPRC_HUMAN | Leucine-rich PPR motif-containing protein, mitochondrial |
| MRPS17 | Q9Y2R5 | RT17_HUMAN | 285 ribosomal protein 517, mitochondrial |
| PARP1 | P09874 | PARP1_HUMAN | Poly [ADP-ribose] polymerase 1 |
| RPF2 | Q9H782 | RPF2_HUMAN | Ribosome production factor 2 homolog |
| HSP90AA1 | P07900 | HS90A_HUMAN | Heat shock protein HSP 90-alpha |
| IGF2BP1 | Q9NZI8 | IF2B1_HUMAN | Insulin-like growth factor 2 mRNA-binding protein 1 |
| DNAJA2 | O60884 | DNJA2 _HUMAN | DnaJ homolog subfamily A member 2 |
| NANOG | Q9H950 | NANOG_HUMAN | Homeobox protein NANOG |
| ALPL | P05186 | PPBT_HUMAN | Alkaline phosphatase, tissue-nonspecific isozyme |
| EIF3B | P55884 | EIF38_HUMAN | Eukaryotic translation initiation factor 3 subunit B |
| NCL | P19338 | NUCL_HUMAN | Nucleolin |
| LIN28A | Q9H9Z2 | LN28A_HUMAN | Protein lin-28 homolog A |
| NOLC1 | Q14978 | NOLC1_HUMAN | Nucleolar and coiled-body phosphoprotein 1 |
| CCT8 | P50990 | TCPQ_HUMAN | T-complex protein 1 subunit theta |
| RRS1 | Q15050 | RRS1_HUMAN | Ribosome biogenesis regulatory protein homolog |
| SFPQ | P23246 | SFPQ_HUMAN | Splicing factor, proline- and glutamine-rich |

On the other hand, it was surprisingly found out that gene expression of L1TD1 correlates with the expression of some other genes in colon cancer. Top 20 of these genes are RETNLB, CLCA1, HEPACAM2, FOXA3, FCGBP, ST6GALNAC1, SPINK4, KIAA1324, KLF4, GMDS, SLITRK6, SERPINA1, LINC00261, ITLN1, MUC2, DEFA5, ASRGL1, SLC27A2, RNF186, and PCCA (Table 2).

TABLE 2
Top 20 co-expressed genes

| GENE NAME | UNIOPROT ID (HUMAN) | UNIPROT ENTRY NAME | UNIPROT PROTEIN NAME |
|---|---|---|---|
| RETNLB | Q9BQ08 | RETNB_HUMAN | Resistin-like beta |
| CLCA1 | A8K714 | CLCA1_HUMAN | Calcium-activated chloride channel regulator 1 |
| HEPACAM2 | ABMVWS | HECA2_HUMAN | HEPACAM family member 2 |
| FOXA3 | PS5318 | FOXA3_HUMAN | Hepatocyte nuclear factor 3-gamma |
| FCGBP | Q9V6R7 | FCGBP_HUMAN | IgGFc-binding protein |
| ST6GALNAC1 | Q9NSC7 | SIA7A_HUMAN | Alpha-N-acetylgalactosaminide alpha-2,6-sialyltransferase 1 |
| SPINK4 | O60575 | ISK4_HUMAN | Serine protease inhibitor Kazal-type 4 |
| KIAA1324 | Q6UXG2 | K1324_HUMAN | UPF0S77 protein KIAA1324 |
| KLF4 | O43474 | KLF4_HUMAN | Krueppel-like factor 4 |
| GMDS | O60547 | GMDS_HUMAN | GDP-mannose 4,6 dehydratase |
| SLITRK6 | Q9H5Y7 | SLIK6_HUMAN | SLIT and NTRK-like protein 6 |
| SERPINA1 | P01009 | A1AT_HUMAN | Alpha-1-antitrypsin |
| LINC00261 | — | — | Long Intergenic Non-Protein Coding RNA 261 |
| ITLN1 | Q8WWA0 | ITLN1_HUMAN | Intelectin-1 |
| MUC2 | Q02817 | MUC2_HUMAN | Mucin-2 |
| DEFA5 | Q01523 | DEF5_HUMAN | Defensin-5 |
| ASRGL1 | Q7L266 | ASGL1_HUMAN | Isoaspartyl peptidase/L-asparaginase |
| SLC27A2 | O14975 | S27A2_HUMAN | Very long-chain acyl-CoA synthetase |
| RNF186 | Q9NXI6 | RN186_HUMAN | RING finger protein 186 |
| PCCA | P05165 | PCCA_HUMAN | Propionyl-CoA carboxylase alpha chain |

Accordingly, the present invention provides a method of prognosing colon cancer in a subject on the basis of the expression level of L1TD1. The method comprises assaying a sample obtained from said subject for the level of L1TD1 expression, and comparing the assayed level of L1TD1 to a control level, and prognosing said colon cancer on the basis of said comparison. In accordance with the present invention, increased expression of L1TD1 indicates good prognosis, whereas decreased or normal expression of L1TD1 indicates poor prognosis.

In some embodiments, the method may further comprise assaying said sample also for one or more interaction partners of L1TD1 selected from the group consisting of OCT4, TRIM71, DPPA4, DNMT3B, LRPPRC, MRPS17, PARP1, RPF2, HSP90AA1, IGF2BP1, DNAJA2, NANOG, ALPL, EIF3B, NCL, LIN28A, NOLC1, CCT8, RRS1, and SFPQ, wherein lack of co-expression with L1TD1 is indicative of good prognosis.

In some further embodiments, preferred interaction partners whose lack of co-expression with L1TD1 is indicative good prognosis, especially prolonged disease-free survival, include OCT4, DNMT3B, NANOG, and LIN28A. Preferred biomarker combinations to be analyzed include L1TD1 and OCT4; L1TD1, OCT4 and DNMT3B; L1TD1, OCT4, NANOG and LIN28A; or L1TD1, OCT4, DNMT3B, NANOG, and LIN28A, wherein lack of co-expression between L1TD1 and the indicated interaction partners is indicative of good prognosis.

Alternatively or in addition, the present method may further comprise assaying said sample also for one or more biomarkers encoded by genes selected from the group consisting of RETNLB, CLCA1, HEPACAM2, FOXA3, FCGBP, ST6GALNAC1, SPINK4, KIAA1324, KLF4, GMDS, SLITRK6, SERPINA1, LINC00261, ITLN1, MUC2, DEFA5, ASRGL1, SLC27A2, RNF186, and PCCA, wherein co-expression with L1TD1 is indicative of good prognosis. Non-limiting examples of preferred biomarker combinations for use in the present invention include the following:

L1TD1 and SPINK4;

L1TD1 and RETNLB;

L1TD1 and ASRGL1;

L1TD1 and CLCA1;

L1TD1 and FCGBP;

L1TD1, SPINK4 and RETNLB;

L1TD1, SPINK4 and ASRGL1;

L1TD1, SPINK4 and CLCA1;

L1TD1, SPINK4 and FCGBP;

L1TD1, RETNLB and ASRGL1;

L1TD1, RETNLB and CLCA1;

L1TD1, RETNLB and FCGBP;

L1TD1, ASRGL1 and CLCA1;

L1TD1, ASRGL1 and FCGBP;

L1TD1, CLCA1 and FCGBP;

L1TD1, SPINK4, RETNLB and ASRGL1;

L1TD1, SPINK4, RETNLB and CLCA1;

L1TD1, SPINK4, RETNLB and FCGBP;

L1TD1, SPINK4, ASRGL1 and CLCA1;

L1TD1, SPINK4, ASRGL1 and FCGBP;

L1TD1, SPINK4, CLCA1 and FCGBP;

L1TD1, RETNLB, ASRGL1 and CLCA1;

L1TD1, RETNLB, ASRGL1 and FCGBP;

L1TD1, RETNLB, CLCA1 and FCGBP;

L1TD1, ASRGL1, CLCA1 and FCGBP;

L1TD1, SPINK4, RETNLB, ASRGL1 and CLCA1;

L1TD1, SPINK4, RETNLB, ASRGL1 and FCGBP;

L1TD1, SPINK4, RETNLB, CLCA1 and FCGBP;

L1TD1, SPINK4, ASRGL1, CLCA1 and FCGBP;

L1TD1, RETNLB, ASRGL1, CLCA1 and FCGBP; and

L1TD1, SPINK4, RETNLB, ASRGL1, CLCA1 and FCGBP.

In some embodiments, particularly potent biomarkers indicative of good prognosis, when co-expressed with L1TD1, include ASRGL1, RETNLB and SPINK4 combinations. Thus, preferred biomarker combination for use in the present invention include L1TD1 and at least one of ASRGL1, RETNLB and SPINK4, especially L1TD1 in combination with ASRGL1, L1TD1 in combination with ASRGL1 and RETNLB, as well as L1TD1 in combination with ASRGL1, RETNLB and SPINK4.

In some embodiments of the present invention, biomarkers indicative of good prognosis, especially when co-expressed with L1TD1, comprise one or more biomarkers encoded by genes selected from the group consisting of RETNLB, FOXA3, SPINK4, DEFA5 and RNF186. Non-limiting examples of preferred biomarker combination, in addition to the ones mentioned above, include the following:

RETNLB;

FOXA3;

SPINK4;

DEFA5;

RNF186;

L1TD1 and RETNLB;

L1TD1 and FOXA3;

L1TD1 and SPINK4;

L1TD1 and DEFA5;

L1TD1 and RNF186;

RETNLB and FOXA3;

RETNLB and SPINK4;

RETNLB and DEFA5;

RETNLB and RNF186;

FOXA3 and SPINK4;

FOXA3 and DEFA5;

FOXA3 and RNF186;

SPINK4 and DEFA5;

SPINK4 and RNF186;

DEFA5 and RNF186;

L1TD1, RETNLB and FOXA3;

L1TD1, RETNLB and SPINK4;

L1TD1, RETNLB and DEFA5;

L1TD1, RETNLB and RNF186;

L1TD1, FOXA3 and SPINK4;

L1TD1, FOXA3 and DEFA5;

L1TD1, FOXA3 and RNF186;

L1TD1, SPINK4 and DEFA5;

L1TD1, SPINK4 and RNF186;

L1TD1, DEFA5 and RNF186;

RETNLB, FOXA3 and SPINK4;

RETNLB, FOXA3 and DEFA5;

RETNLB, FOXA3 and RNF186;

RETNLB, SPINK4 and DEFA5;

RETNLB, SPINK4 and RNF186;

RETNLB, DEFA5 and RNF186;

FOXA3, SPINK4 and DEFA5;

FOXA3, SPINK4 and RNF186;

FOXA3, DEFA5 and RNF186;

SPINK4, DEFA5 and RNF186;

L1TD1, RETNLB, FOXA3 and SPINK4;

L1TD1, RETNLB, FOXA3 and DEFA5;

L1TD1, RETNLB, FOXA3 and RNF186;

L1TD1, RETNLB, SPINK4 and DEFA5;

L1TD1, RETNLB, SPINK4 and RNF186;

L1TD1, RETNLB, DEFA5 and RNF186;

L1TD1, FOXA3, SPINK4 and DEFA5;

L1TD1, FOXA3, SPINK4 and RNF186;

L1TD1, FOXA3, DEFA5 and RNF186;

L1TD1, SPINK4, DEFA5 and RNF186;

RETNLB, FOXA3, SPINK4 and DEFA5;

RETNLB, FOXA3, SPINK4 and RNF186;

RETNLB, FOXA3, DEFA5 and RNF186;

RETNLB, SPINK4, DEFA5 and RNF186;

FOXA3, SPINK4, DEFA5, and RNF186;

L1TD1, RETNLB, FOXA3, SPINK4 and DEFA5;

L1TD1, RETNLB, FOXA3, SPINK4 and RNF186;

L1TD1, RETNLB, FOXA3, DEFA5 and RNF186;

L1TD1, RETNLB, SPINK4, DEFA5 and RNF186;

L1TD1, FOXA3, SPINK4, DEFA5 and RNF186;

RETNLB, FOXA3, SPINK4, DEFA5 and RNF186; and

L1TD1, RETNLB, FOXA3, SPINK4, DEFA5 and RNF186.

The present invention also provides a method of prognosing colon cancer in a subject, wherein said method comprises assaying a sample obtained from said subject for the expression level of one or more biomarkers encoded by genes selected from the group consisting of L1TD1, RETNLB, CLCA1, HEPACAM2, FOXA3, FCGBP, ST6GALNAC1, SPINK4, KIAA1324, KLF4, GMDS, SLITRK6, SERPINA1, LINC00261, ITLN1, MUC2, DEFA5, ASRGL1, SLC27A2, RNF186, and PCCA, and comparing the assayed level of said one or more biomarkers to a control level, and prognosing said colon cancer on the basis of said comparison. Preferably, increased expression of said one or more biomarkers is indicative of good prognosis. Non-limiting examples of preferred biomarkers and biomarker combinations for use in the present invention, in addition to the ones listed above, include the following:

SPINK4;

RETNLB;

ASRGL1;

CLCA1;

FCGBP;

SPINK4 and RETNLB;

SPINK4 and ASRGL1;

SPINK4 and CLCA1;

SPINK4 and FCGBP;

RETNLB and ASRGL1;

RETNLB and CLCA1;

RETNLB and FCGBP;

ASRGL1 and CLCA1;

ASRGL1 and FCGBP;

CLCA1 and FCGBP;

SPINK4, RETNLB and ASRGL1;

SPINK4, RETNLB and CLCA1;

SPINK4, RETNLB and FCGBP;

SPINK4, ASRGL1 and CLCA1;

SPINK4, ASRGL1 and FCGBP;

SPINK4, CLCA1 and FCGBP;

RETNLB, ASRGL1 and CLCA1;

RETNLB, ASRGL1 and FCGBP;

RETNLB, CLCA1 and FCGBP;

ASRGL1, CLCA1 and FCGBP;

SPINK4, RETNLB, ASRGL1 and CLCA1;

SPINK4, RETNLB, ASRGL1 and FCGBP;

SPINK4, RETNLB, CLCA1 and FCGBP;

SPINK4, ASRGL1, CLCA1 and FCGBP;

RETNLB, ASRGL1, CLCA1 and FCGBP; and

SPINK4, RETNLB, ASRGL1, CLCA1 and FCGBP.

As used herein, the term “prognosis” refers to a probable course or clinical outcome of a disease, while the expressions “prognosticating”, “prognosing”, “determining a prognosis”, and the like, refer to a prediction of future progression of colon cancer.

As used herein, terms “good prognosis” and “positive prognosis” refer to a probable statistically significantly prolonged survival, such as prolonged overall survival, prolonged disease-free survival, prolonged recurrence-free survival, or prolonged progression-free survival as compared to the median outcome of the disease or to survival in subjects with poor prognosis.

As used herein, term “poor prognosis” refers to a probable statistically significantly reduced survival, such as reduced overall survival, disease-free survival, recurrence-free survival or progression-free survival than in subjects with good prognosis.

In accordance with the present invention, the prognosis is made on the basis of detected levels of L1TD1, which associates with the prognosis of colon cancer, in a biological sample obtained from the subject whose colon cancer is to be prognosed. This is also meant to include instances where the prognosis is not finally determined but that further testing is warranted. In such embodiments, the method is not by itself determinative of the prognosis of a subject's colon cancer but can indicate that further testing is needed or would be beneficial. Therefore, the present method may be combined with one or more other methods for the final determination of the prognosis. Such other methods are well known to a person skilled in the art, including but not limited to, colonoscopy, biopsy, molecular characterization of the tumor, computed tomography scan, magnetic resonance imaging, and positron emission tomography scan, and monitoring levels of Carcinoembryonic antigen (CEA). Additional predictive markers that may be used in combination with the present invention include, but are not limited to, RAS (KRAS and NRAS) mutations, BRAF mutations, molecular profiling of tumors, examining chromosomal stability of tumors (microsatellite stable (MSS) and microsatellite instable (MSI)).

As used herein, the term “subject” refers to mammals such as humans and domestic animals such as livestock, pets, and sporting animals. Examples of such animals include without limitation carnivores such as cats and dogs and ungulates such as horses. As used herein, the terms “subject” and “individual” are interchangeable.

As used herein, the term “sample” refers to a biological sample, typically a clinical sample, and encompasses, for example, blood and other bodily fluids including, but not limited to, peripheral blood, serum, plasma, urine, and saliva; and solid tissue samples such as biopsy specimens, especially those comprising cancerous cells. In certain embodiments, blood samples such as serum or plasma samples are the most preferred sample types to be used in the present method. Generally, obtaining the sample to be analyzed from a subject is not part of the present prognostication method.

The term “sample” also includes samples that have been manipulated or treated in any appropriate way after their procurement, including but not limited to centrifugation, filtration, precipitation, dialysis, chromatography, treatment with reagents, washing, or enriching for a certain component of the sample such as a cell population.

As used herein, the terms “biomarker” and “marker” are interchangeable, and refer to a molecule that is differentially present in a sample taken from subjects suffering from colon cancer with good prognosis, as compared to a comparable sample take from control subjects, such as subjects suffering from colon cancer with poor prognosis. Thus, the present biomarkers provide information regarding a probable course of colon cancer and associate with the positive prognosis of colon cancer. The term “present biomarker” refers to any individual biomarker set forth above, preferably L1TD1, or to any biomarker combination thereof. Thus, the term encompasses not only L1TD1 but also any combinations of L1TD1 and one or more of its interaction partners set for above and/or one or more biomarkers set forth above that are co-expressed with L1TD1.

Herein, the term “level”, when applied to a biomarker, is used inter-changeably with the terms “amount” and “concentration”, and can refer to an absolute or relative quantity of the biomarker.

As used herein, the term “control” may refer to a comparable sample obtained from a control subject or a pool of control subjects with a known colon cancer history or no history. Appropriate control subjects include individuals who are apparently healthy, and thus, do not show any signs of colon cancer. In some embodiments, preferred control subjects are individuals or pools of individuals who have a colon cancer with poor prognosis. In some further embodiments, subjects or pools of subjects who have colon cancer with good prognosis may be employed as appropriate control subjects. Sometimes it may be beneficial to use more than one type of controls in a single prognostication method.

The term “control” may also refer to a predetermined threshold or control value, originating from a single control subject or a pool of control subjects set forth above, which value is indicative of the prognosis of colon cancer. Statistical methods for determining appropriate threshold or control values will be readily apparent to those of ordinary skill in the art, and the statistically validated threshold or control values can take a variety of forms. For example, a statistically validated threshold can be a single cut-off value, such as a median or mean. Alternatively, a statistically validated threshold can be divided equally (or unequally) into groups, such as low, medium, and high risk groups, the low-risk group being individuals least likely to have aggressive colon cancer and the high-risk group being individuals most likely to develop aggressive colon cancer with short survival time. Furthermore, the threshold may be an absolute value or a relative value. However, if an absolute value is used for the level of the assayed biomarker, then the threshold value is also based upon an absolute value. The same applies to relative values, which must be comparable. In some embodiments, the biomarker levels are normalized using standard methods prior to being compared with a relevant control.

In some embodiments, subjects of the same age, demographic features, and/or disease status, etc. may be employed as appropriate control subjects for obtaining comparable control samples or determining a statistically validated threshold value.

The levels of the assayed biomarkers in the patient sample may be compared with one or more single control values or with one or more ranges of control values, regardless of whether the control value is a predetermined value or a value obtained from a control sample upon practicing the prognostication method. The significance of the difference of biomarker levels in the patient sample and the control can be assessed using standard statistical methods. In some embodiments, of the present invention, a statistically significant increase between the assayed biomarker level and a negative control level indicates that the patient is more likely to have good prognosis than an individual with biomarker levels comparable to the statistically validated negative control value. In such cases, increased biomarker levels are indicative of good prognosis of colon cancer. On the other hand, a statistically significant non-increase between the assayed biomarker level and a negative control level indicates that the patient is not likely to have a good prognosis or indicate that the patient has a poor prognosis. Furthermore, a statistically significant non-increase between the assayed biomarker level and a positive control level indicates that the patient is likely to have a good prognosis.

As used herein, expressions like “indicative of good prognosis of colon cancer” refer, at least in some embodiments, to a biomarker which, using routine statistical methods setting confidence levels at a minimum of 95%, is prognostic for colon cancer such that the biomarker is found significantly more often, or in higher levels, in subjects with good outcome of colon cancer than in subjects with poor outcome. Preferably, a prognostic biomarker which is indicative of a good prognosis is found in at least 80% of subjects with prolonged colon cancer-associated survival, and is found in less than 10% of subjects with reduced colon cancer-associated survival. More preferably, a prognostic biomarker which is indicative of good prognosis is found in at least 90%, at least 95%, at least 98%, or more in subjects with prolonged colon cancer-associated survival and is found in less than 10%, less than 8%, less than 5%, less than 2.5%, or less than 1% of subjects with reduced colon cancer-associated survival.

As used herein, the term “increased level” refers to an increase in the amount of a biomarker in a sample as compared with a relevant control. Said increase can be determined qualitatively and/or quantitatively according to standard methods known in the art. The term “increased” encompasses an increase at any level, but refers more specifically to an increase between about 10% and about 250% as compared with a relevant control. In some embodiments, the biomarker is increased by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, 160%, by at least 170%, by at least 180%, by at least 190%, by at least 200%, by at least 250%, or more. In some embodiments, the term “increased level” refers to a statistically significant increase in the level or amount of the biomarker as compared with that of a relevant control.

As used herein, the term “non-increased” or “normal” refers to a detected or assayed biomarker level that is essentially the same or essentially non-altered as compared with that of a relevant control sample or a predetermined threshold value.

In some embodiments, the prognosis may be based on analyzing one or more serial samples obtained from the subject, for example, to detect any changes in the prognosis, and may involve a prediction of or monitoring for a response to a particular treatment or combination of treatments for colon cancer. In such instances, the prognostication method comprises analyzing and comparing at least two samples obtained from the same subject at various time points. The number and interval of the serial samples may vary as desired. The difference between the obtained assessment results serves as an indicator of the progression of colon cancer or as an indicator of effectiveness or ineffectiveness of the treatment or combination of treatments applied.

In some embodiments, the present method of prognosing colon cancer may include monitoring for or characterization of the tumor, for example, based on anatomical site, histological subtype, T stage (invasion), N (regional lymph node metastasis), M (distant metastatis), circumferential margin (only rectum), mesorectal intactness (only rectum), histological response to neoadjuvant treatment (only rectum), vascular invasion, Lymphatic invasion, Perineural invasion, Grade, Tumour budding, Perforation. Also envisaged is monitoring for progression or response to treatment, by imaging (computed tomography scan, magnetic resonance imaging, and positron emission tomography scan), and analyzing circulating tumor markers, etc.

The present method of prognosing colon cancer in an individual may be used not only for determining, predicting or monitoring an individual's risk of or progression towards colon cancer but also for screening new therapeutics for colon cancer. It is envisaged that L1TD1 may be used for assessing whether or not a candidate drug or intervention therapy is able to increase the expression level of L1TD1 of a subject with poor prognosis towards that of a positive control or towards that of an individual who has good prognosis of colon cancer. Furthermore, individuals identified to have a poor prognosis of colon cancer on the basis of their non-increased L1TD1 expression level could be employed as targets in clinical trials aimed for identifying new therapeutic drugs or other intervention therapies for colon cancer. Thus, L1TD1 may also be used for stratifying individuals for clinical trials.

In some implementations, the present method of prognosing colon cancer in a subject having colon cancer may further include therapeutic intervention. Once a subject is identified to have a given probable outcome of the disease, he/she may be subjected to an appropriate therapeutic intervention, such as chemotherapy. In such implementations, the invention may also be formulated as a method of treating colon cancer in a subject in need thereof, wherein the method comprises prognosing colon cancer as set forth above, and administering one or more appropriate chemotherapeutic agents to said subject.

The expression level of any one of the present biomarkers may be determined by a variety of techniques. In particular, the expression at the nucleic acid level may be determined by measuring the quantity of RNA, preferably mRNA or any other RNA species representing the biomarker in question, using methods well known in the art. Non-limiting examples of suitable methods include digital PCR and real-time (RT) quantitative or semi-quantitative PCR. Primers suitable for these methods may be easily designed by a skilled person.

Further suitable techniques for determining the expression level of any one of the present biomarkers at nucleic acid level include, but are not limited to, fluorescence-activated cell sorting (FACS) and in situ hybridization.

Other non-limiting ways of measuring the quantity of RNA, preferably mRNA or any other RNA species representing the biomarker in question, include transcriptome approaches, in particular, DNA microarrays. Generally, when it is the quantity of mRNA that is to be determined, test and control mRNA samples are reverse transcribed and labeled to generate cDNA probes. The probes are then hybridized to an array of complementary nucleic acids immobilized on a solid support. The array is configured such that the sequence and position of each member of the array is known. Hybridization of a labeled probe with a particular array member indicates that the sample from which the probe was derived expresses that gene. Non-limiting examples of commercially available microarray systems include Affymetrix GeneChip™ and Illumina BeadChip.

Furthermore, bulk RNA sequencing, single-cell RNA sequencing or cDNA sequencing, e.g. by Next Generation Sequencing (NGS) methods, may also be used for determining the expression level of any one of the present biomarkers.

If desired, the quantity of RNA, preferably mRNA any other RNA species representing the biomarker in question, may also be determined or measured by conventional hybridization-based assays such as Northern blot analysis, as well as by mass cytometry.

Changes in the regulation of activity of a gene encoding the biomarker in question can be determined through epigenetic analysis, such as histone modification analysis, for example by chromatin immunoprecipitation followed by sequencing or quantitative PCR, or quantitation of DNA methylation levels, for example by bisulfite sequencing or capture based methods, at the intergenic regulatory sites or gene region of the biomarker in question.

As is readily apparent to a skilled person, a variety of techniques may be employed for determining the expression level of any one of the present biomarkers at the protein level. Non-limiting examples of suitable methods include mass spectrometry-based quantitative proteomics techniques, such as isobaric Tags for Relative and Absolute Quantification reagents (iTRAQ) and label-free analysis, as well as selected reaction monitoring (SRM) mass spectrometry and any other techniques of targeted proteomics. Also, the level or amount of a protein marker may be determined by e.g. an immunoassay (such as ELISA or LUMINEX®), Western blotting, spectrophotometry, an enzymatic assay, an ultraviolet assay, a kinetic assay, an electro-chemical assay, a colorimetric assay, a turbidimetric assay, an atomic absorption assay, flow cytometry, mass cytometry, or any combination thereof. Further suit-able analytical techniques include, but are not limited to, liquid chromatography such as high performance/pressure liquid chromatography (HPLC), gas chromatography, nuclear magnetic resonance spectrometry, related techniques and combinations and hybrids thereof, for example, a tandem liquid chromatography-mass spectrometry (LC-MS).

The present disclosure also relates to an in vitro kit for prognosing colon cancer in a subject. The kit may be used in any implementation of the present method or its embodiments. At minimum, the kit comprises one or more testing agents or reagents that are capable of detecting one or more of the present biomarkers, preferably at least L1TD1, or determining its expression level.

In some embodiments, the kit may comprise a pair of primers and/or a probe specific to L1TD1. A skilled person can easily design suitable primers and/or probes taking into account specific requirements of a technique to be applied. The kit may further comprise means for detecting the hybridization of the probes with nucleotide molecules, such as mRNA or cDNA, representing L1TD1 in a test sample and/or means for amplifying and/or detecting the nucleotide molecules representing L1TD1 in the test sample by using the pairs of primers.

In some embodiments, the kit may also comprise one or more testing agents or reagents for detecting one or more genes co-regulated with L1TD1 or interaction partners of L1TD1 in accordance with the disclosure above.

Other optional components in the kit include a compartmentalized carrier means, one or more buffers (e.g. block buffer, wash buffer, substrate buffer, etc.), other reagents, positive or negative control samples, etc.

The kit may also comprise a computer readable medium comprising computer-executable instructions for performing any method of the present disclosure.

It will be obvious to a person skilled in the art that, as technology advances, the inventive concept can be implemented in various ways. The invention and its embodiments are not limited to the examples described below but may vary within the scope of the claims.

### Materials and Methods

### Microarray Data Sets

Raw microarray data sets were downloaded from Gene Expression Omnibus (GEO). Three colon cancer gene expression microarray data sets comprising a total of 1052 clinical samples were analyzed. Either due to a non-tumoral origin (i.e. normal tissue) or due to missing associated survival information, 124 samples had to be excluded from the survival analysis (928 samples remained). A summary of the data sets used is presented in Table 3. Additionally, two seminoma and one stem cell gene expression microarray data sets were analyzed to assess the co-expression of L1TD1 and its interaction partners (Table 1). It is noteworthy that the stem cell data set “hESC1” was not a homogenous hESC data set, instead it was composed of samples from ten hESCs, 49 induced pluripotent stem cells, five cancer cell lines, and six non-cancerous somatic cell lines.

TABLE 3
Summary of the data sets used in the study
The table lists the GEO accession numbers together with the alias names
which are used to refer to these individual data sets, the microarray
platform, and the number of samples used in the analyses.

| | Total | Survival | | |
|---|---|---|---|---|
| GEO ID | Samples | Analysis | Platform | Alias |
| GSE14333 | 290 | 226 | Affymetrix HG-U133Plus2 | colon1 |
| GSE17536 | 177 | 145 | Affymetrix HG-U133Plus2 | colon2 |
| GSE39582 | 585 | 557 | Affymetrix HG-U133Plus2 | Colon3 |
| GSE3218 | 107 | Not used | Affymetrix HG-U133A | seminoma1 |
| GSE10783 | 34 | Not used | Affymetrix HG-U133A | seminoma2 |
| GSE42445 | 70 | Not used | Agilent-028004 SurePrint G3 | hESC1 |
| | | | Human GE 8x60K | |

### Gene Expression Analysis

The CEL files, containing the probe intensity measurements of the Affymetrix probes were normalized using the Universal exPression Code (UPC) normalization method from the Bioconductor package “SCAN.UPC” and the Robust Multiarray Average (RMA) normalization method from the Bioconductor package “affy”. The UPC normalization method provides a score between 0.0 and 1.0, which represents the probability that a particular gene is expressed in a particular sample. The UPC scores were used to categorize the samples in all data sets based on their L1TD1 expression status as L1TD1 high (UPC>=0.60) and L1TD1 low (UPC<0.60). The probe “219955_at” was chosen as the primary probe for the quantification of L1TD1 because it was present in both of the Affymetrix platforms used in this study (HGU133 plus 2.0, and HG-U133A). RMA provides normalized log 2 intensity values. RMA normalized gene expression values were used to calculate pairwise correlations between genes.

### Survival Analysis of Microarray Data

Disease-free survival was analyzed in each data set with the Kaplan-Meier method as implemented in the R package “survival” and survival curves were plotted using the R package “survminer”. The log-rank test was used to compare survival rates between the two L1TD1 groups (high L1TD1 and low L1TD1). A total of 928 samples with complete information about survival time and survival status were included in the analysis.

### Results

### A Subset of Colon Cancer Patients Express L1TD1 at High Levels

Our results show that 26.7% of colon cancer patients fall into the L1TD1-high group, which is in agreement with immunohistochemistry data available from the Human Protein Atlas (Table 4). However, the proportion of L1TD1-high samples was lower in colon cancer, in comparison to seminoma (48.6% and 50.0%) and hESCs (88.6%) (Table 4).

TABLE 4
Proportion of samples with high expression of L1TD1
The table shows categorization of samples based on their L1TD1 expression
status in the different data sets used in this study. For colon cancer data sets,
only tumor samples with complete survival information were considered.

| | | | | Percentage of |
|---|---|---|---|---|
| Dataset | LITD1+ | L1TD1− | Total | L1TD1 + |
| colon1 | 64 | 162 | 226 | 28.3% |
| colon2 | 44 | 101 | 145 | 30.3% |
| colon3 | 140 | 417 | 557 | 25.1% |
| Total (Colon Cancer) | 248 | 680 | 928 | 26.7% |
| seminoma1 | 52 | 55 | 107 | 48.6% |
| seminoma2 | 17 | 17 | 34 | 50.0% |
| hESC1 | 62 | 8 | 70 | 88.6% |

High Levels of L1TD1 Associate with Longer Disease-Free Survival

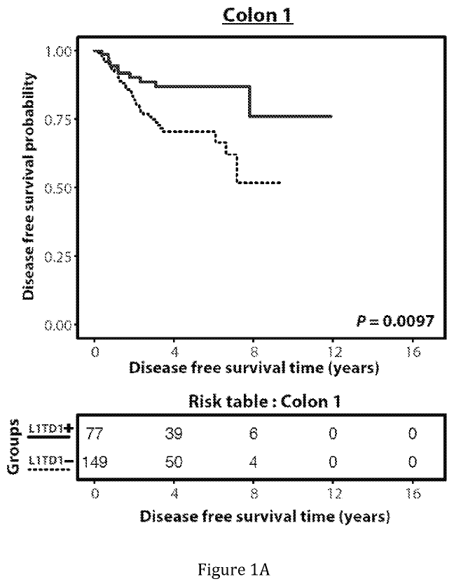

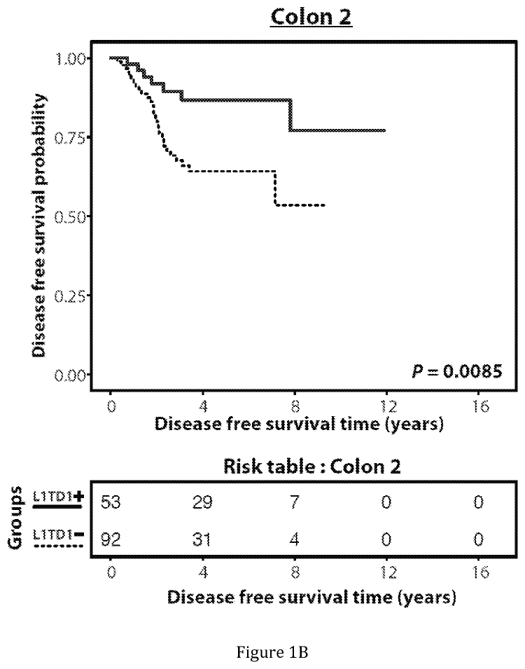

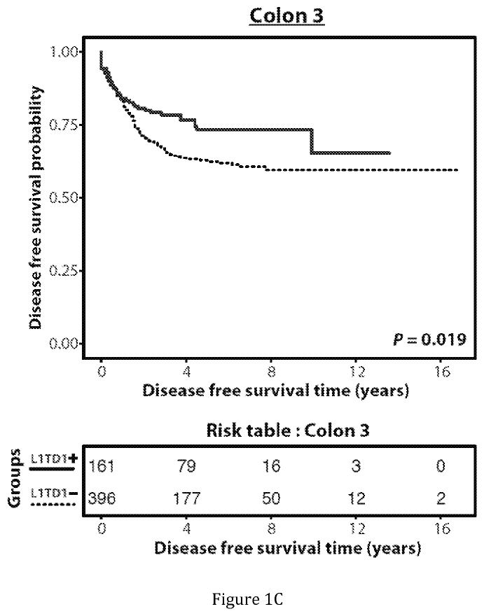

Kaplan-Meier analysis of 928 samples with associated survival information from the three colon cancer data sets revealed that the L1TD1-high colon cancer group had longer disease-free survival as compared to those with no/low L1TD1 expression (FIGS. 1A-1C). The difference was significant in all of the three data sets (P<0.05).

### Interactome of L1TD1 is not Co-Expressed in Colon Cancer

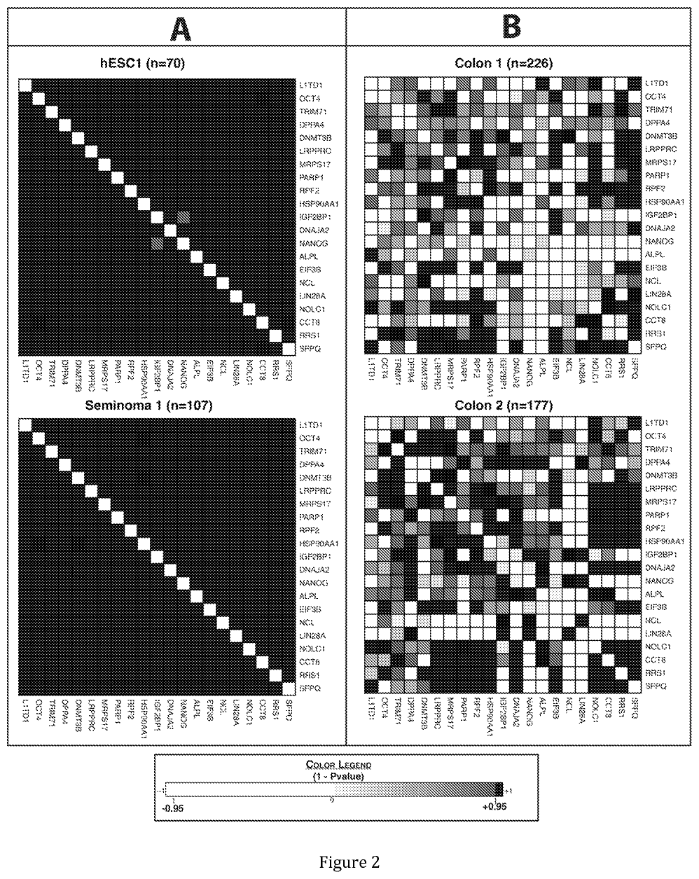

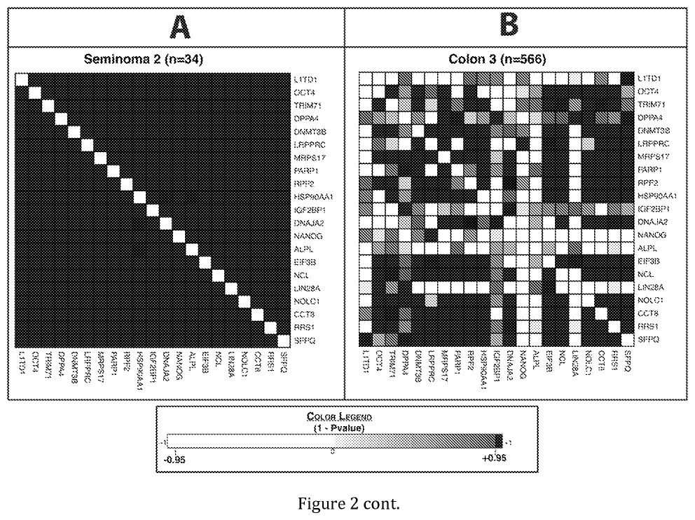

To examine the potential role of the known interaction partners [311 Interaction partners of L1TD1 were determined using Mass spectrometry and co-immunoprecipitation in our earlier publication (Emani, Närvä et. al., Stem Cell Reports, 2015)] of L1TD1 in the different prognostic behavior of L1TD1 in colon cancer, Spearman rank correlation matrices were calculated between the expression levels of L1TD1 and its interaction partners. A high positive correlation (correlation value >0.5 and P<0.0001) was observed among L1TD1 and its top 20 interaction partners in seminoma and in the stem cell data sets (FIG. 2A). Conversely, all three of the colon cancer data sets lacked correlation among these genes and L1TD1 (FIG. 2B).

Genes Co-Expressed with L1TD1 in Colon Cancer

We also identified other genes that were co-expressed with L1TD1 in colon cancer patients. For each colon cancer data set, the genes were ranked (best gene gets the smallest rank) based on the descending order of the Spearman rank correlation score. For each gene, its maximum rank (worst rank) among the three data sets was taken as its final rank. The list was sorted in ascending order of the maximum rank of each gene and top 20 genes were selected (Table 5).

TABLE 5
Top 20 positively correlated genes with L1TD1 in colon
cancer data sets
Statistical significance of correlation is represented using circles that
correspond to false discovery rate (FDR) value ranges. The top genes
were selected by ranking all the genes in the microarray datasets
separately for each colon cancer data set based on Spearman
rank correlation scores for pairwise correlation between L1TD1
and each gene. Then, the maximum rank over the colon cancer data
sets was selected as a representative statistic for each gene. The
list was ordered (ascending) based on this maximum rank, and 20
genes were selected from the top of the list.

| Rank | Gene Name | Colon 1 | Colon 2 | Colon 3 |
|---|---|---|---|---|
| 1 | RETNLB | 0.47 * | 0.53 * | 0.45 * |
| 2 | CLCA1 | 0.45 * | 0.43 + | 0.45 * |
| 3 | HEPACAM2 | 0.43 * | 0.41 + | 0.46 * |
| 4 | FOXA3 | 0.41 * | 0.43 + | 0.43 * |
| 5 | FCGBP | 0.41 * | 0.39 + | 0.47 * |
| 6 | ST6GALNAC1 | 0.40 * | 0.39 + | 0.43 * |
| 7 | SPINK4 | 0.44 * | 0.38 + | 0.43 * |
| 8 | KIAA1324 | 0.40 * | 0.44 + | 0.39 * |
| 9 | KLF4 | 0.40 * | 0.37 + | 0.41 * |
| 10 | GMDS | 0.46 * | 0.40 + | 0.38 * |
| 11 | SLITRK6 | 0.43 * | 0.36 x | 0.46 * |
| 12 | SERPINA1 | 0.42 * | 0.38 + | 0.35 * |
| 13 | LINC00261 | 0.34 + | 0.35 x | 0.48 * |
| 14 | ITLN1 | 0.35 * | 0.33 x | 0.42 * |
| 15 | MUC2 | 0.39 * | 0.33 x | 0.38 * |
| 16 | DEFA5 | 0.37 * | 0.35 x | 0.33 * |
| 17 | ASRGL1 | 0.40 * | 0.32 x | 0.41 * |
| 18 | SLC27A2 | 0.36 * | 0.36 + | 0.33 * |
| 19 | RNF186 | 0.32 + | 0.36 x | 0.34 * |
| 20 | PCCA | 0.37 * | 0.37 + | 0.33 * |

Significance threshold:
* FDR < 0.000001
+ 0.001 > FDR > 0.000001
x 0.05 > FDR > 0.001
∘ FDR > 0.05

Table 6 below lists the top 20 genes that are co-expressed with L1TD1 in colon cancer, along with the P-values showing their impact on survival in colon cancer patients, when tested individually. Five genes, namely SPINK4, RETNLB, ASRGL1, CLCA1 and FCGBP, were statistically significant in two out of three datasets.

TABLE 6

| | Gene | Colon1 | Colon2 | Colon3 |
|---|---|---|---|---|
| Colon1 | L1TD1 | 0.009729 | 0.008520 | 0.018607 |
| 1 | SPINK4 | 0.007148 | 0.001854 | 0.880992 |
| 2 | RETNLB | 0.325642 | 0.012519 | 0.009064 |
| 3 | ASRGL1 | 0.015986 | 0.521116 | 0.016293 |
| 4 | CLCA1 | 0.030053 | 0.006496 | 0.710961 |
| 5 | FCGBP | 0.028617 | 0.047080 | 0.292182 |
| 6 | ITLN1 | 0.088225 | 0.043802 | 0.844453 |
| 7 | FOXA3 | 0.077752 | 0.609721 | 0.093598 |
| 8 | PCCA | 0.064797 | 0.601176 | 0.107992 |
| 9 | DEFA5 | 0.136904 | 0.157008 | 0.737800 |
| 10 | GMDS | 0.318171 | 0.170255 | 0.000919 |
| 11 | HEPACAM2 | 0.368837 | 0.687066 | 0.098125 |
| 12 | SERPINA1 | 0.000008 | 0.493419 | 0.911649 |
| 13 | RNF186 | 0.700045 | 0.541107 | 0.010793 |
| 14 | KLF4 | 0.938136 | NA | 0.220231 |
| 15 | ST6GALNAC1 | 0.593332 | 0.880638 | 0.030027 |
| 16 | MUC2 | 0.624983 | 0.505661 | 0.842770 |
| 17 | KIAA1324 | 0.220079 | 0.969530 | 0.730810 |
| 18 | SLITRK6 | 0.750696 | 0.894483 | 0.085490 |
| 19 | LINC00261 | 0.823520 | 0.823442 | 0.269044 |
| 20 | SLC27A2 | 0.883481 | 0.975288 | 0.002906 |

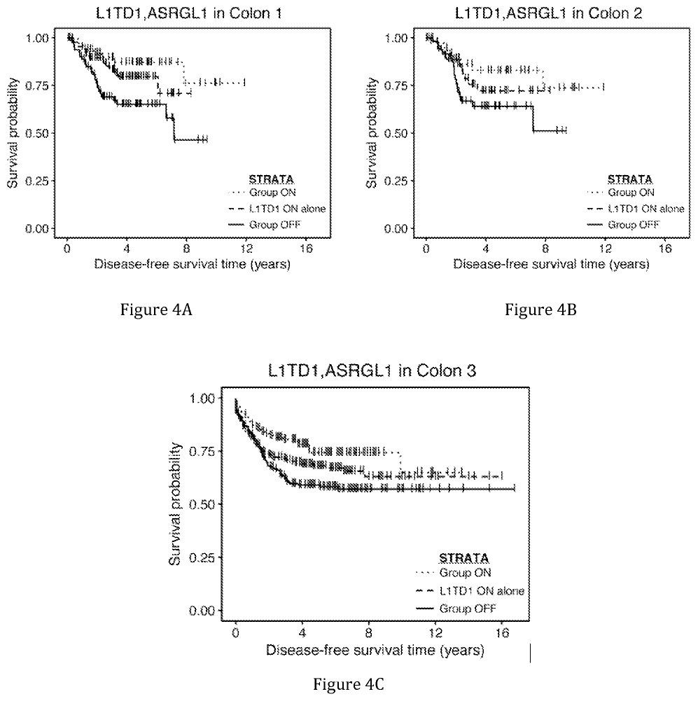

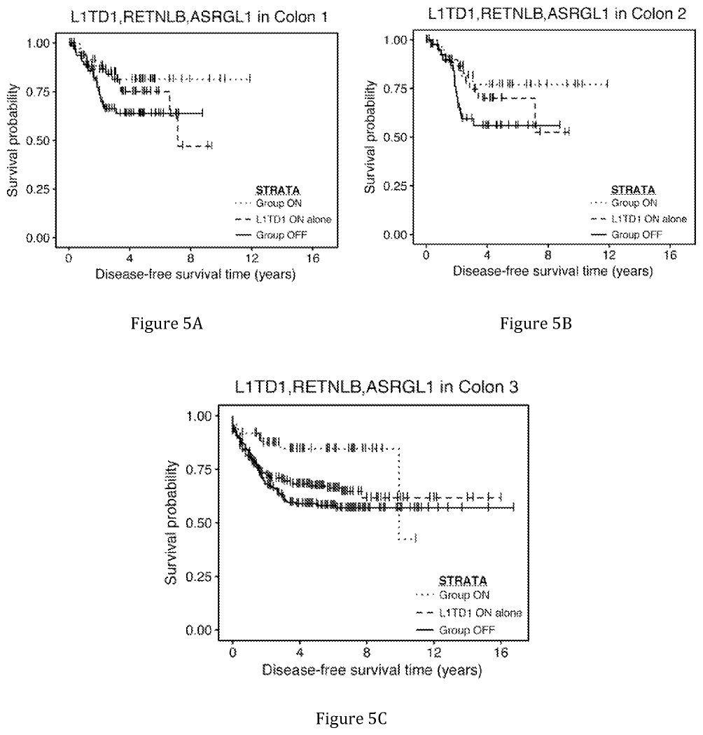

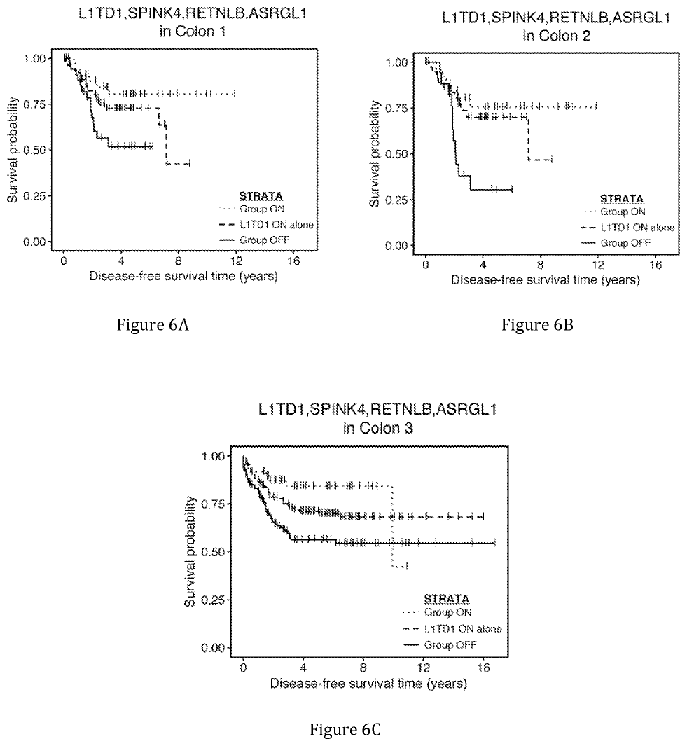

Although, none of the top 20 co-expressed genes (listed in Table 2) outperformed L1TD1 as independent prognostic marker for colon cancer in all the three data sets, five genes had statistically significant (P<0.05) impact on survival in at least two out of the three colon cancer data sets: SPINK4, RETNLB, ASRGL1, CLCA1, FCGBP. When we added this additional information for stratifying the samples, combinations of L1TD1 and the co-expressed genes were identified that predicted survival even better than L1TD1 alone, including L1TD1+ASRGL1, in L1TD1+ASRGL1+RETNLB, and L1TD1+ASRGL1+RETNLB+SPINK4 (FIGS. 4A-6C).

The performance of these combinations in the three data sets were compared to each other by using weighted ranks to prioritize the combinations. Initially, for each data set combinations which performed better than L1TD1 alone in the three data sets received a lower rank (i.e. 1=best). Using the ranks from the three data sets, a weighted rank was computed (weight=number of samples in the data set/Total samples in the study (928)) to summarize the performance of combinations. Based on these results, marker combination L1TD1+ASRGL1+RETNLB performed the best, followed by the marker combination L1TD1+ASRGL1, and then by the marker combination L1TD1+ASRGL1+RETNLB+SPINK4.

### Discussion

In this study, we found compelling evidence of L1TD1 being a positive prognostic marker for colon cancer (FIGS. 1A-1C). We demonstrated this by survival analysis of 928 samples from three gene expression data sets which were comprised of 1052 colon cancer patients. However, increased expression of L1TD1 in combination with increased expression of ASRGL1; ASRGL1 and RETNLB; or ASRGL1, RETNLB and SPINK4 was an even stronger indicator of prolonged disease-free survival.

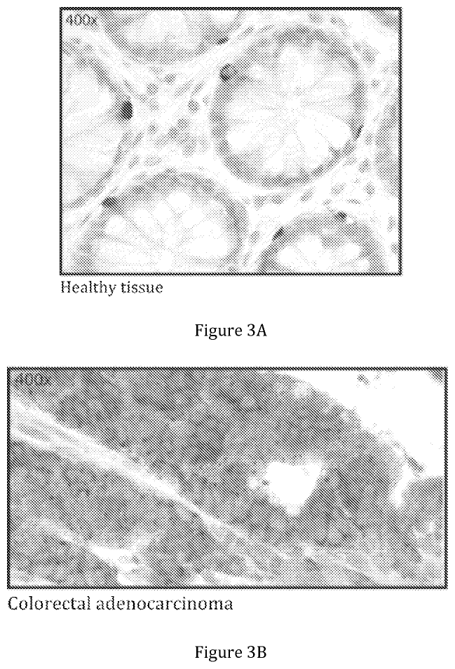

Expression of L1TD1 has earlier been reported to be highly specific to embryonic stem cells, brain, and colon (FIG. 3A). Besides these, L1TD1 has also been reported to be expressed in seminoma, embryonic carcinomas, medulloblastoma, and colon adenocarcinoma (FIG. 3B). Expression of L1TD1 at high levels in colon cancer cells led us to hypothesize that high expression of L1TD1 in colon cancer might be associated with prognosis. Earlier reports have demonstrated the association of OCT4 & NANOG with poor prognosis in different cancer types, including medulloblastoma and seminoma. Interestingly, our results were in contrast with previous studies, suggesting that in colon cancer, high expression of L1TD1 is linked to better prognosis.

In an attempt to investigate the distinctive role of L1TD1 in different cancers, we investigated the co-expression of L1TD1 with its currently-known interaction partners. We discovered that, unlike in hESCs and seminomas, L1TD1 was not co-expressed with its interaction partners in colon cancer (FIG. 2). This points to the potential participation of L1TD1's interaction partners in the contrasting prognostic outcome. This was further supported by a recent study in medulloblastoma, showing an association of high L1TD1 expression with poor clinical outcome and significant co-expression between L1TD1 and its interaction partner, OCT4. Together, these findings suggest that the co-expression of L1TD1 with its interaction partners might be required for manifesting an aggressive and detrimental phenotype. This is the first time that an embryonic stem cell factor has been shown to lead to contrasting outcomes in cancer, taking into consideration the presence or absence of strong co-expression with its interaction partners.

Our analysis of gene expression data from three clinical colon cancer data sets produced promising evidence in support of L1TD1, especially in combination with a further biomarker selected from ASRGL1, RETNLB and SPINK4, in as a marker for good prognosis in colon cancer.

1-12. (canceled)

13. A method of prognosing colon cancer in a subject, wherein the method comprises
assaying a sample obtained from said subject for the level of LINE-1 type transposase domain containing 1 (L1TD1) and isoaspartyl peptidase/L-asparaginase (ASRGL1) expression, and
comparing the assayed levels of L1TD1 and ASRGL1 to respective control levels, and
prognosing said colon cancer on the basis of said comparison, wherein increased expression of L1TD1 and ASRGL1 is indicative of good prognosis.

14. The method according to claim 13, wherein the method further comprises assaying said sample for the level of resistin-like beta (RETNLB), wherein co-expression with L1TD1 and ASRGL1 is indicative of good prognosis.

15. The method according to claim 14, wherein the method further comprises assaying said sample for the level of serine protease inhibitor Kazal-type 4 (SPINK4), wherein co-expression with L1TD1, ASRGL1 and RETNLB is indicative of good prognosis.

16. The method according to claim 15, further comprising assaying said sample also for one or more biomarkers encoded by genes selected from the group consisting of CLCA1, HEPACAM2, FOXA3, FCGBP, ST6GALNAC1, KIAA1324, KLF4, GMDS, SLI-TRK6, SERPINA1, LINC00261, ITLN1, MUC2, DEFA5, SLC27A2, RNF186 and PCCA, wherein co-expression with L1TD1, ASRGL1, RETNLB and SPINK4 is indicative of good prognosis.

17. The method according to claim 13, further comprising assaying said sample for one or more biomarkers selected from the group consisting of OCT4, TRIM71, DPPA4, DNMT3B, LRPPRC, MRPS17, PARP1, RPF2, HSP90AA1, IGF2BP1, DNAJA2, NANOG, ALPL, EIF3B, NCL, LIN28A, NOLC1, CCT8, RRS1, and SFPQ, wherein lack of co-expression with L1TD1 is indicative of good prognosis.

18. The method according to claim 13, wherein said sample is selected from the group consisting of a peripheral blood sample, a serum sample, a plasma sample, an urine sample, a saliva sample, and a tissue sample.

19. The method according to claim 13, for determining, predicting or monitoring an individual's risk of or progression towards colon cancer, stratifying individuals for clinical trials and screening new therapeutics for colon cancer.

20. Use of a biomarker combination defined in claim 13 for prognosing colon cancer.

21. A kit for use in the method according to claim 13, wherein the kit comprises one or more testing agents capable of specifically detecting the expression level of L1TD1 and ASRGL1 in a biological sample obtained from a subject whose colon cancer is to be prognosed.

22. The kit according to claim 21, wherein the kit further comprises one or more testing agents capable of specifically detecting the expression level of one or more biomarkers selected from the group consisting of CLCA1, HEPACAM2, FOXA3, FCGBP, ST6GALNAC1, KIAA1324, KLF4, GMDS, SLI-TRK6, SERPINA1, LINC00261, ITLN1, MUC2, DEFA5, SLC27A2, RNF186 and PCCA, wherein co-expression with L1TD1, ASRGL1, RETNLB and SPINK4 is indicative of good prognosis, or one or more biomarkers selected from the group consisting of OCT4, TRIM71, DPPA4, DNMT3B, LRPPRC, MRPS17, PARP1, RPF2, HSP90AA1, IGF2BP1, DNAJA2, NANOG, ALPL, EIF3B, NCL, LIN28A, NOLC1, CCT8, RRS1, and SFPQ, wherein lack of co-expression with L1TD1 is indicative of good prognosis and/or genes selected from the genes listed in Table 1 or Table 2.

23. Use of a kit for carrying out the method according to claim 13, wherein the kit comprises one or more testing agents capable of specifically detecting the expression level of L1TD1 and ASRGL1 in a biological sample obtained from a subject whose colon cancer is to be prognosed.

24. The use of a kit according to claim 23, wherein the kit further comprises one or more testing agents capable of specifically detecting the expression level of one or more biomarkers selected from the group consisting of CLCA1, HEPACAM2, FOXA3, FCGBP, ST6GALNAC1, KIAA1324, KLF4, GMDS, SLI-TRK6, SERPINA1, LINC00261, ITLN1, MUC2, DEFA5, SLC27A2, RNF186 and PCCA, wherein co-expression with L1TD1, ASRGL1, RETNLB and SPINK4 is indicative of good prognosis, or one or more biomarkers selected from the group consisting of OCT4, TRIM71, DPPA4, DNMT3B, LRPPRC, MRPS17, PARP1, RPF2, HSP90AA1, IGF2BP1, DNAJA2, NANOG, ALPL, EIF3B, NCL, LIN28A, NOLC1, CCT8, RRS1, and SFPQ, wherein lack of co-expression with L1TD1 is indicative of good prognosis and/or genes selected from the genes listed in Table 1 or Table 2.
